(19) United States
(12) Patent Application Publication
(10) Pub. No.: US 2023/0337748 A1
(43) Pub. Date: Oct. 26, 2023

(54) VAPORIZER AND ELECTRONIC VAPORIZATION DEVICE

(71) Applicant: Jiangmen Moore Technology., Ltd, Jiangmen (CN)

(72) Inventors: Zhouwei CHEN, Jiangmen (CN); Chao ZHANG, Jiangmen (CN); Guanghui LI, Jiangmen (CN)

(21) Appl. No.: 18/343,343

(22) Filed: Jun. 28, 2023

Related U.S. Application Data
(63) Continuation of application No. PCT/CN2021/143303, filed on Dec. 30, 2021.

(30) Foreign Application Priority Data
Dec. 30, 2020 (CN) 202011611873.8

(51) Int. Cl. A24F 40/485; A24F 40/10

(52) U.S. Cl. CPC A24F 40/485; A24F 40/10; A24F 40/46

(57) ABSTRACT

A vaporizer includes: a vaporization sleeve having a liquid storage cavity and a mounting cavity; a mounting base arranged in the mounting cavity and forming a vaporization cavity, an air inlet channel and an air outlet channel being in communication with the vaporization cavity; a vaporization core mounted in the vaporization cavity; and a fine groove provided on an inner wall of the air inlet channel, the fine groove buffering liquid entering the air inlet channel.

### CROSS-REFERENCE TO PRIOR APPLICATION

This application is a continuation of International Patent Application No. PCT/CN2021/143303, filed on Dec. 30, 2021, which claims priority to Chinese Patent Application No. 202011611873.8, filed on Dec. 30, 2020. The entire disclosure of both applications is hereby incorporated by reference herein.

### FIELD

This application relates to the technical field of electronic vaporization devices, and in particular, to a vaporizer and an electronic vaporization device.

### BACKGROUND

In the related art, an electronic vaporization device generally includes a vaporizer and a power supply assembly. A liquid storage cavity and a vaporization cavity are generally provided in the vaporizer. The liquid storage cavity is configured to store a vaporizable medium, and a vaporization assembly may be arranged in the vaporization cavity for heating and vaporizing the vaporizable medium to form vapor that can be inhaled by an inhaler; and the power supply component is configured to supply power to the vaporizer.

An air inlet channel and an air outlet channel that are in communication with the vaporization cavity further need to be arranged in the vaporizer, and the air outlet channel may be in communication with a suction nozzle for the inhaler to inhale vapor in the vaporization cavity; and the air inlet channel may communicate the vaporization cavity with the external air, so that when the inhaler inhales, the external air enters the vaporization cavity through the air inlet channel. In this way, vapor generated by vaporization of the vaporizer is driven by an airflow and delivered to the inhaler.

The existing electronic vaporization device is prone to a problem of leakage of condensate of a vaporization medium from the air inlet channel after being used for a period of time.

### SUMMARY

In an embodiment, the present invention provides a vaporizer, comprising: a vaporization sleeve having a liquid storage cavity and a mounting cavity; a mounting base arranged in the mounting cavity and forming a vaporization cavity, an air inlet channel and an air outlet channel being in communication with the vaporization cavity; a vaporization core mounted in the vaporization cavity; and a fine groove provided on an inner wall of the air inlet channel, the fine groove being configured to buffer liquid entering the air inlet channel.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an embodiment of a vaporizer according to this application;

FIG. 2 is a schematic structural diagram of an exploded view of the vaporizer shown in FIG. 1;

FIG. 3 is a cross-sectional view of the vaporizer shown in FIG. 1;

FIG. 4 is a schematic structural diagram of the base shown in FIG. 2;

FIG. 5 is a schematic structural diagram of the base shown in FIG. 4 from another perspective;

FIG. 6 is a schematic structural diagram of an embodiment of the seal member shown in FIG. 2;

FIG. 7 is a cross-sectional view of the seal member shown in FIG. 6; and

FIG. 8 is a schematic structural diagram of an embodiment of an electronic vaporization device according to this application.

### DETAILED DESCRIPTION

In an embodiment, the present invention provides an electronic vaporization device and a vaporizer thereof, to resolve the foregoing technical problem.

In an embodiment, the present invention provides a vaporizer, including:
a vaporization sleeve, where the vaporization sleeve has a liquid storage cavity and a mounting cavity;
a mounting base, where the mounting base is arranged in the mounting cavity, and forms a vaporization cavity and an air inlet channel and an air outlet channel that are in communication with the vaporization cavity; and
a vaporization core, where the vaporization core is mounted in the vaporization cavity; and
a fine groove is provided on an inner wall of the air inlet channel, and the fine groove is configured to buffer liquid entering the air inlet channel.

Optionally, the mounting base includes a base and a seal member, and the seal member is arranged on a side of the base close to the liquid storage cavity, and is engaged with the base and surrounds the base to form the vaporization cavity.

Optionally, an outer surface of a side wall of the base has an air guide groove, the air guide groove cooperates with a side wall of the mounting cavity to form the air inlet channel, an end of the air inlet channel close to the liquid storage cavity is in communication with the vaporization cavity, and an end away from the liquid storage cavity is in communication with external air; and

the fine groove is provided in the air guide groove.

Optionally, the air guide groove extends along a side of the mounting base close to the liquid storage cavity to a side away from the liquid storage cavity, and the fine groove is provided on a bottom wall of the air guide groove and the width of the fine groove ranges from 0.2 mm to 1.2 mm.

Optionally, the air guide groove extends along a side of the mounting base close to the liquid storage cavity to a side away from the liquid storage cavity, and the fine groove is provided on a bottom wall of the air guide groove and the width of the fine groove ranges from 0.3 mm to 0.7 mm.

Optionally, a side of the air guide groove close to the liquid storage cavity has a first intercepting groove, and the fine groove is in communication with the vaporization cavity through the first intercepting groove.

Optionally, a side of the air guide groove away from the liquid storage cavity has a second intercepting groove, and the fine groove is in communication with the external air through the second intercepting groove.

Optionally, a middle portion of the air guide groove has at least one third intercepting groove, and the third intercepting groove separates the fine groove.

Optionally, the first intercepting groove is a first notch on an edge of the side wall of the base close to the liquid storage cavity, and the second intercepting groove is a second notch on an edge of a bottom wall of the mounting base.

Optionally, a baffle plate is arranged between the air inlet channel and the vaporization cavity;
the baffle plate closes the air inlet channel in a natural state, so as to disconnect communication between the air inlet channel and the vaporization cavity; and
the baffle plate opens the air inlet channel when an inhaler inhales air from the vaporization cavity through the air outlet channel, so as to communicate the air inlet channel with the vaporization cavity.

Optionally, a side wall of the seal member has an engagement groove, and the engagement groove is sleeved on a side wall of the base; and a side wall on a side of the engagement groove is arranged between the air inlet channel and a side wall of the mounting cavity, and a side wall on an other side of the engagement groove is arranged between the vaporization core and the side wall of the base.

Optionally, the baffle plate is fixedly connected to a bottom wall of the engagement groove of the seal member, and the baffle plate is integrally formed with the seal member.

Optionally, a cross section of the vaporization cavity has two opposite long sides and two opposite short sides, and the air inlet channel is located on either side of the long sides.

To resolve the foregoing technical problem, a technical solution adopted in this application is to provide an electronic vaporization device, including:
a vaporizer, where the vaporizer is configured to store to-be-vaporized liquid and vaporize the to-be-vaporized liquid to form an aerosol for inhalation by an inhaler, and the vaporizer is the vaporizer as described above; and
a body assembly, where the body assembly is configured to supply power to the vaporizer.

Beneficial effects of this application are as follows: this application provides an electronic vaporization device and a vaporizer thereof. By providing a fine groove in an air inlet channel, condensed vaporized liquid in the air inlet channel may enter the fine groove for buffering, thereby preventing the condensed vaporized liquid from leaking out from an air inlet of the air inlet channel. Further, by providing the air inlet channel on either side of long sides of a cross section of a vaporization cavity, when a vaporization core is mounted in the vaporization cavity, a journey of passing a vaporization surface of the vaporization core performed by an airflow entering the vaporization cavity from the air inlet channel may be lengthened, thereby increasing a heating time of the airflow. In this way, condensate of vaporized liquid and splashed vaporized liquid generated in a vaporizer may be fully absorbed or heated and vaporized by the vaporization core, reducing occurrence of a phenomenon of inhalation leakage and condensate accumulation.

In order to make the objects, technical solutions and advantages of embodiments of this application clearer, technical solutions of the embodiments of this application will be clearly and comprehensively described in the following with reference to the accompanying drawings in the embodiments of this application. It is apparent that the described embodiments are a part of the embodiments of this application, rather than all of the embodiments. All other embodiments obtained by a person skilled in the art based on the embodiments of this application without creative efforts shall fall within the protection scope of this application.

In this application, the terms “first”, “second” and “third” are used merely for the purpose of description, and shall not be construed as indicating or implying relative importance or implying a quantity of indicated technical features. Therefore, features defining “first” “second” and “third” can explicitly or implicitly include at least one of the features. In description of this application, “more” means at least two, such as two and three unless it is specifically defined otherwise. All directional indications (for example, up, down, left, right, front, back) in the embodiments of this application are only used for explaining relative position relationships, movement situations, or the like between the various components in a specific posture (as shown in the accompanying drawings). If the specific posture changes, the directional indications change accordingly. In addition, the terms “include”, “have”, and any variant thereof are intended to cover a non-exclusive inclusion. For example, a process, method, system, product, or device that includes a series of steps or units is not limited to the listed steps or units; and instead, further optionally includes a step or unit that is not listed, or further optionally includes another step or unit that is intrinsic to the process, method, product, or device.

“Embodiment” mentioned in the specification means that particular features, structures, or characteristics described with reference to the embodiment may be included in at least one embodiment of this application. The term appearing at different positions of the specification may not refer to the same embodiment or an independent or alternative embodiment that is mutually exclusive with another embodiment. A person skilled in the art explicitly or implicitly understands that the embodiments described in the specification may be combined with other embodiments in the absence of conflict. Detailed descriptions are provided below respectively with reference to specific embodiments.

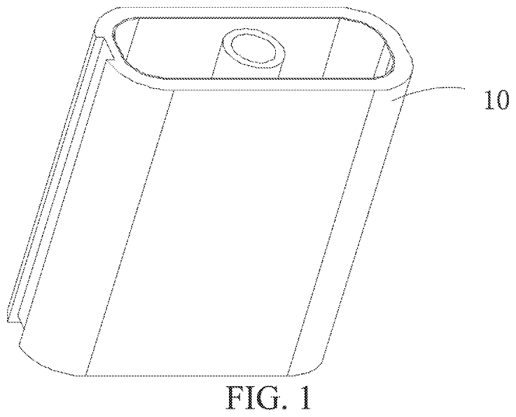

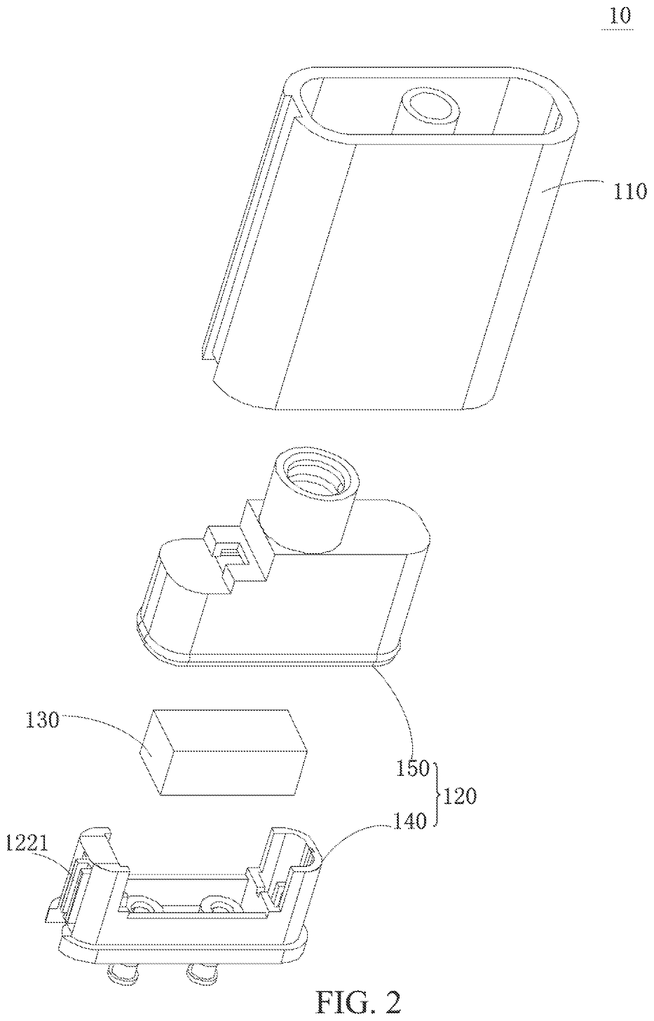

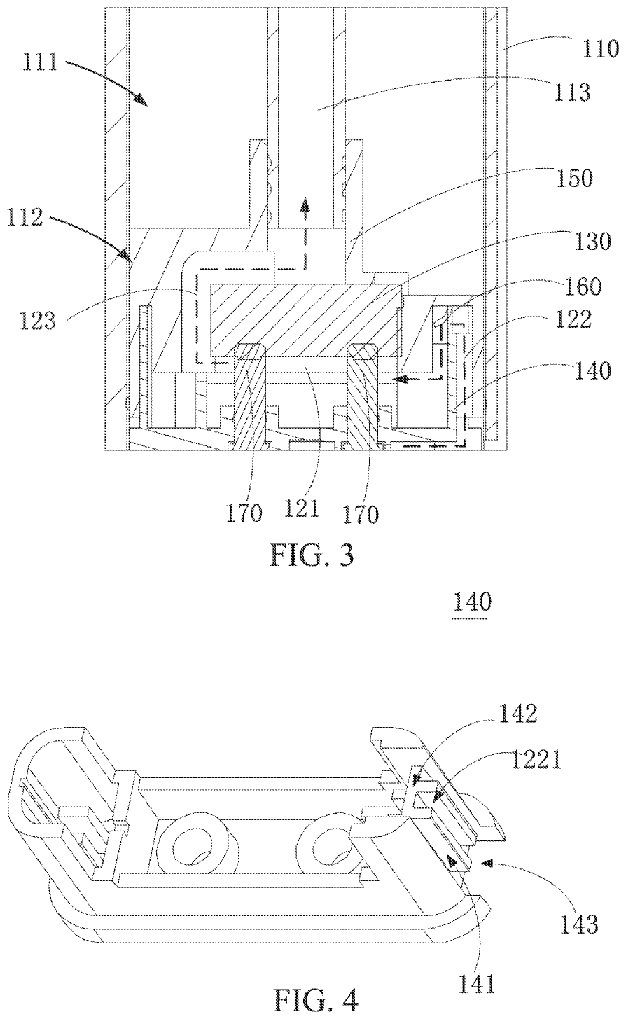

Referring to FIG. 1 to FIG. 3, FIG. 1 is a schematic structural diagram of an embodiment of a vaporizer according to this application, and FIG. 2 is a schematic structural diagram of an exploded view of the vaporizer shown in FIG. 1; and FIG. 3 is a cross-sectional view of the vaporizer shown in FIG. 1.

A vaporizer 10 includes a vaporization sleeve 110, a mounting base 120, and a vaporization core 130.

The vaporization sleeve 110 has a liquid storage cavity 111 and a mounting cavity 112; and the mounting base 120 is arranged in the mounting cavity 112, and forms a vaporization cavity 121 and an air inlet channel 122 and an air outlet channel 123 that are in communication with the vaporization cavity 121. A vaporization core 130 is mounted in the vaporization cavity 121, and may be in communication with the liquid storage cavity 111, so that the vaporization core 130 in the vaporization cavity 121 may receive vaporized liquid stored in the liquid storage cavity 111 and heat and vaporize the vaporized liquid. In this way, the vaporized vapor (or aerosol) of the vaporized liquid may be formed in the vaporization cavity 121.

The air inlet channel 122 may communicate the vaporization cavity 121 with external air, and may be configured to supply air to the vaporization cavity 121; and the air outlet channel 123 may be in communication with a preset suction nozzle. When an inhaler inhales air through the air outlet channel 123, air in the vaporization cavity 121 may be sucked out so that air pressure in the vaporization cavity 121 is lower than the atmospheric pressure. Through the action of a pressure difference, the external air may enter the vaporization cavity 121 from the air inlet channel 122, and be sucked out through the air outlet channel 123, thereby forming an airflow, and the vaporized vapor of the vaporized liquid may be sucked out with the movement of the airflow.

When the inhaler stops inhaling air, air backflow may occur in the vaporization cavity 121, so that the vapor in the vaporization cavity 121 enters the air inlet channel 122. The vapor entering the air inlet channel 122 condenses to form vaporized liquid, and may leak out from an air inlet of the air inlet channel 122. In this embodiment, by providing a fine groove 1221 on an inner wall of an air inlet channel 122, condensed vaporized liquid in the air inlet channel 122 may enter the fine groove 1221 for buffering, thereby preventing the condensed vaporized liquid from leaking out from an air inlet of the air inlet channel 122.

The vaporized liquid may be absorbed under surface tension of the vaporized liquid and stored in the fine groove 1221, and the width of the fine groove 1221 may be set to range from 0.2 mm to 1.2 mm, for example, may be set to 0.2 mm, 0.7 mm, or 1.2 mm. In a preferred implementation, the width of the fine groove 1221 may be set to range from 0.3 mm to 0.7 mm, for example, may be set to 0.3 mm, 0.5 mm, or 0.7 mm.

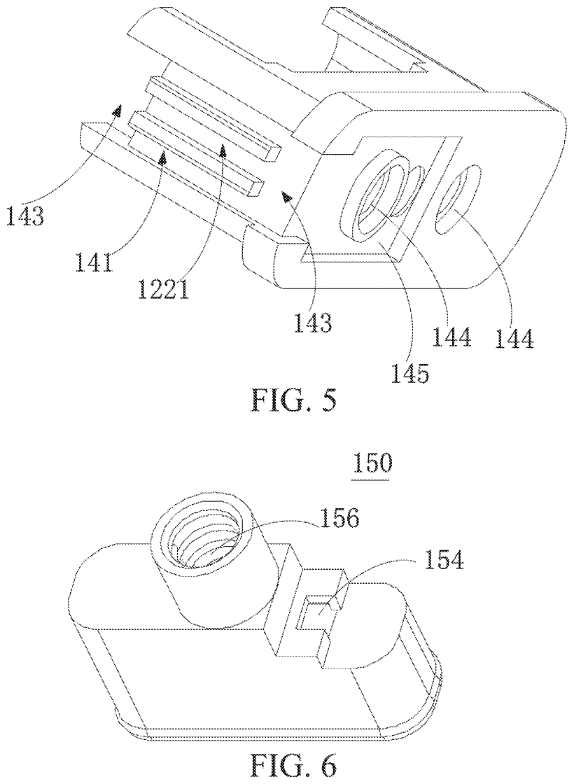

Further, referring to FIG. 2, FIG. 4, and FIG. 5, FIG. 4 is a schematic structural diagram of the base shown in FIG. 2; and FIG. 5 is a schematic structural diagram of the base shown in FIG. 4 from another perspective.

In this embodiment, the mounting base 120 may be at least partially inserted into a vaporization sleeve 110 for sealing the vaporization sleeve 110.

The mounting base 120 includes a base 140 and a seal member 150. The seal member 150 may be arranged on a side of the base 140 close to the liquid storage cavity 111, and is engaged with the base 140 and surrounds the base to form the vaporization cavity 121. An outer surface of a side wall of the base 140 has an air guide groove 141, and the air guide groove 141 cooperates with a side wall of a mounting cavity 112 (or a side wall of the seal member 150) to form the air inlet channel 122; and an end of the air inlet channel 122 close to the liquid storage cavity 111 is in communication with the vaporization cavity 121, and an end away from the liquid storage cavity 111 is in communication with the external air, where the fine groove 1221 may be provided in the air guide groove 141.

In this embodiment, at least two protruding walls spaced apart are arranged in the air guide groove 141, and the two adjacent protruding walls may surround to form the fine groove 1221. In another embodiment, the fine groove 1221 may also be a groove provided on an inner wall of the air guide groove 141.

An extension direction of the fine groove 1221 may be arranged parallel to an extension direction of the air guide groove 141. A quantity of fine grooves 1221 may also be one or at least two, and at least two fine grooves 1221 may be spaced apart in the air guide groove 141.

In this embodiment, the fine groove 1221 may extend from the outer surface of the side wall of the base 140 into the vaporization cavity 121.

A side of the air guide groove 141 close to the liquid storage cavity 111 has a first intercepting groove 142, and the fine groove 1221 is in communication with the vaporization cavity 121 through the first intercepting groove 142.

Optionally, the first intercepting groove 142 may cut off an inner wall of the fine groove 1121 and an inner wall of the vaporization cavity 121; and the first intercepting groove 142 may be a first notch on the side wall of the base 140 close to an edge of the liquid storage cavity 141, and the first intercepting groove 142 may cut off the fine groove 1221, thereby preventing a problem of liquid leakage caused by liquid in the vaporization cavity 121 flowing out along the fine groove 1221 due to capillary action.

A side of the air guide groove 141 away from the liquid storage cavity 111 has a second intercepting groove 143, and the fine groove 1221 may be in communication with the external air through the second intercepting groove 143. In this embodiment, the second intercepting groove 143 is a second notch on an edge of a bottom wall of the mounting base 120, and the second intercepting groove 143 may cut off a connection between the fine groove 1221 and an inner wall on which an air inlet of the external air is located.

In another embodiment, a third intercepting groove may further be provided in the air guide groove 141 on the side wall of the base 140, where the third intercepting groove may further cut off the fine groove 1221 on the side wall of the base 140. A quantity of third intercepting grooves is one or more.

Further, in this embodiment, a cross section of the vaporization cavity 121 may be rectangular or approximately rectangular, and has two opposite long sides and two opposite short sides. The air inlet channel 122 and the air outlet channel 123 may be respectively located on two opposite sides of the long sides. When the vaporization core 130 is mounted in the vaporization cavity 121, long sides of a vaporization surface of the vaporization core 130 may be arranged along the long sides of the cross section of the vaporization cavity 121.

Therefore, by respectively providing the air inlet channel 122 and the air outlet channel 123 on two opposite sides of the long sides of the cross section of the vaporization cavity 121, when a vaporization core 130 is mounted in the vaporization cavity 121, a journey of passing a vaporization surface of the vaporization core 130 performed by an airflow entering the vaporization cavity 121 from the air inlet channel 122 may be lengthened, thereby increasing a heating time of the airflow. In this way, condensate of vaporized liquid and splashed vaporized liquid generated in a vaporizer 10 may be fully absorbed or heated and vaporized by the vaporization core 130, reducing a phenomenon of inhalation leakage and condensate accumulation.

Further, to further improve the sealing effect of the vaporizer 10, and reduce the occurrence of a problem of the liquid leakage of the vaporizer 10, a baffle plate may further be arranged in the first intercepting groove 142.

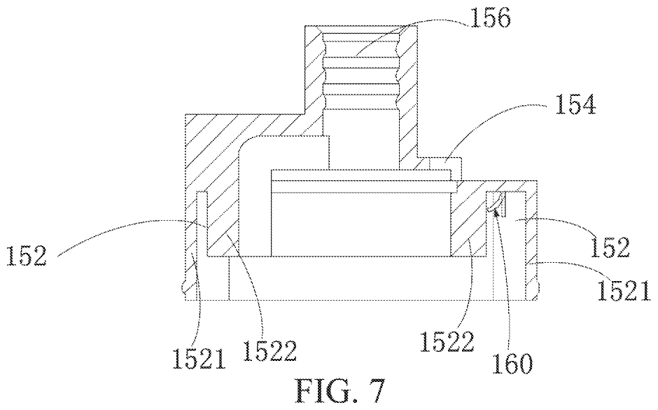

Referring to FIG. 3, FIG. 6 and FIG. 7, FIG. 6 is a schematic structural diagram of an embodiment of the seal member shown in FIG. 2. FIG. 7 is a cross-sectional view of the seal member shown in FIG. 6.

The baffle plate 160 may be arranged in the first intercepting groove 142. The baffle plate 160 may close the first intercepting groove 142 in a natural state, so as to disconnect communication between the air inlet channel 122 and the vaporization cavity 121; and the baffle plate 160 opens the first intercepting groove 142 when an inhaler inhales air from the vaporization cavity 121 through the air outlet channel 123, so as to communicate the air inlet channel 122 with the vaporization cavity 121.

In this embodiment, the baffle plate 160 may be mounted at a joint between the vaporization cavity 121 and the air inlet channel 122, and an opening at the joint between the vaporization cavity 121 and the air inlet channel 122 may be sealed by the baffle plate 160, thereby reducing a risk of the liquid in the vaporization cavity 121 leaking into the air inlet channel 122.

Specifically, the baffle plate 160 may be mounted on a side of the seal member 150 close to the base 140. When the base 140 is connected to the seal member 150, the baffle plate 160 may be partially inserted into the first intercepting groove 142. In addition, when the baffle plate 160 is in a natural state, the baffle plate 160 may abut against or be close to abut against a bottom wall and a side wall of the first intercepting groove 142, so as to separate the first intercepting groove 142.

When the inhaler inhales air from the vaporization cavity 121 through the air outlet channel 123, the air pressure in the vaporization cavity 121 decreases. The baffle plate 160 is bent and deformed under the action of a pressure difference between the air inlet channel 122 and the vaporization cavity 121, to cause the first intercepting groove 142 to be opened, so that the air inlet channel 122 is in communication with the vaporization cavity 121. In this case, the external air may enter the vaporization cavity 121 along the air inlet channel 122, and then be sucked out along the air outlet channel 123. Therefore, an airflow may be generated in the vaporization cavity 121, so that vapor of the vaporized liquid in the vaporization cavity 121 is sucked out from the air outlet channel 123 along with the airflow.

When the inhaler stops inhaling air from the vaporization cavity 121 through the air outlet channel 123, the baffle plate 160 may gradually return to a natural state, to close the first intercepting groove 142. Therefore, liquid in the vaporization cavity 121 (such as condensate of the vaporized liquid) may be prevented from leaking from the air inlet channel 122.

In this embodiment, the baffle plate 160 may be integrally formed with the seal member 150, or may be an independent component with the seal member 150, and assembled after the two are respectively formed.

In another embodiment, the baffle plate 160 may further be mounted on the base 140. Specifically, the baffle plate 160 may be mounted on a bottom wall of the first intercepting groove 142. When the seal member 150 is mounted on the base 140 and is engaged with and fixedly connected to the base 140, the baffle plate 160 may abut against or be close to abut against a bottom wall and a side wall of the seal member 150. In this way, the baffle plate 160 may close the air inlet channel 122 formed by the first intercepting groove 142 and the seal member 150 in a natural state, and disconnect communication between the air inlet channel 122 and the vaporization cavity 121 at a location of the first intercepting groove 142. Reference is further made to FIG. 6 to FIG. 7.

The seal member 150 has an engagement groove 152, and the engagement groove 152 is sleeved on the side wall of the base 140, so that the seal member 150 may be engaged with the side wall of the base 140 through the engagement groove 152, to fixedly connect the base 140 with the seal member 150.

The engagement groove 152 may be provided on the side wall of the seal member 150, and the side wall of the base 140 may be inserted into the engagement groove 152. Specifically, the side wall 1521 on one side of the engagement groove 152 may be arranged between the air guide groove 141 and the side wall of the mounting cavity 112, and cooperate with the air guide groove 141 to form the air inlet channel 122. The side wall 1522 on the other side of the engagement groove 152 is arranged between the vaporization core 130 and the side wall of the base 140.

In this embodiment, the seal member 150 may be formed by a flexible material such as silica gel or rubber.

When the mounting base 120 is mounted in the mounting cavity 112 of the vaporization sleeve 110, the seal member 150 may fill a gap between the base 140 and an inner wall of the vaporization sleeve 110, so as to seal a location of the mounting cavity 112 of the vaporization sleeve 110.

A liquid feeding channel 154 and an air outlet pipeline 156 are further provided on a side of the seal member 150 close to the liquid storage cavity 111 of the vaporization sleeve 110. The liquid feeding channel 154 may be docked with the liquid storage cavity 111. When the vaporization core 130 is arranged in the vaporization cavity 121, the vaporization core 130 may be covered on an opening of the liquid feeding channel 154 located in the vaporization cavity 121, so that a liquid absorbing surface of the vaporization core 130 is at least partially exposed to the liquid storage cavity 111 through the liquid feeding channel 154, and the liquid absorbing surface of the vaporization core 130 is in contact with the vaporized liquid stored in the liquid storage cavity 111. Therefore, the vaporized liquid in the liquid storage cavity 111 may be heated and vaporized by the vaporization core 130 to form vapor of the vaporized liquid in the vaporization cavity 121.

One end of the air outlet pipeline 156 may be docked with an inhalation pipeline 113 in the vaporization sleeve 110, and the other end of the air outlet pipeline 156 may be in communication with the air outlet channel 123. That is, vapor formed into the vaporized liquid in the vaporization cavity 121 may be sucked out from the inhalation pipeline 113 along the air outlet channel 123 and an air outlet pipeline 156.

Reference may be further made to FIG. 3 and FIG. 5, an electrode hole 144 may be further provided on a side of the bottom wall of the base 140. The electrode hole 144 is configured to arrange a conductive member 170, and one end of the conductive member 170 may be inserted into the vaporization cavity 121 through the electrode hole 144 to be electrically connected to the vaporization core 130 therein. The other end of the conductive member 170 may be exposed from a side of the bottom wall of the base 140 through the electrode hole 144. The other end of the conductive member 170 may be electrically connected to an external power supply, to supply power to the vaporization core 130, so that the vaporization core 130 heats and vaporizes the vaporized liquid to form vapor of the vaporized liquid in the vaporization cavity 121.

Further, a bottom air inlet groove 145 is further provided on the bottom wall of the base 140, where the bottom air inlet groove 145 may be in communication with the external air. An air guide groove 141 on the side wall of the base 140 may extend to be in communication with the bottom air inlet groove 145.

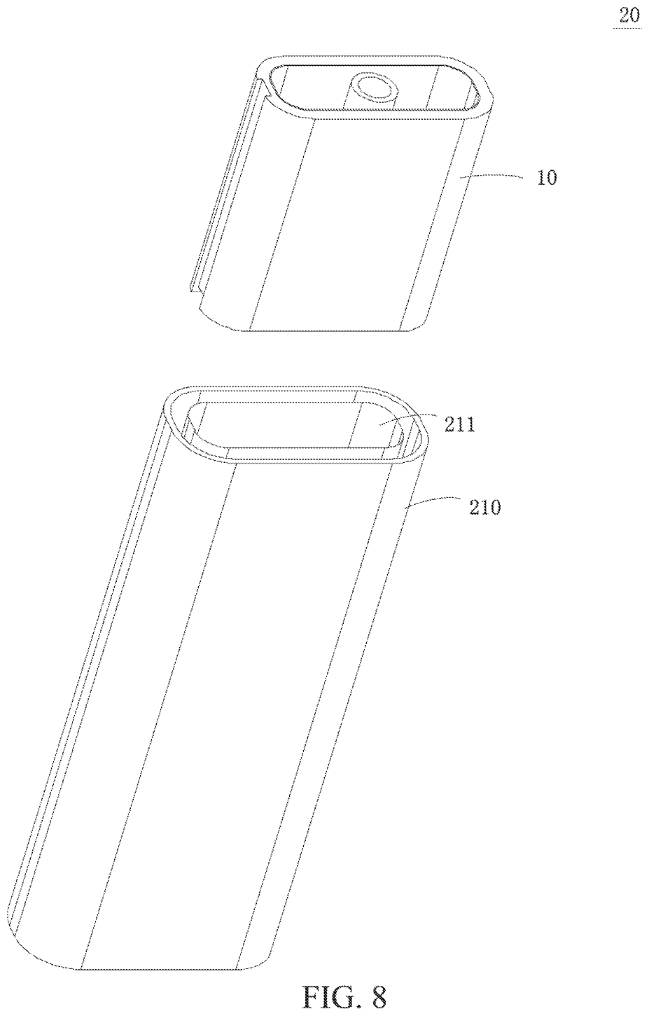

Further, this application further provides an electronic vaporization device. FIG. 8 is a schematic structural diagram of an embodiment of an electronic vaporization device according to this application.

The electronic vaporization device 20 includes a vaporizer 10 and a body assembly 210. The vaporizer 10 may be configured to store the vaporized liquid and vaporize the vaporized liquid to form an aerosol that may be inhaled by the inhaler. The vaporizer 10 may be mounted on the body assembly 210, and a power supply assembly is arranged in the body assembly 210. When the vaporizer 10 is mounted on the body assembly 210, positive and negative poles of the power supply assembly in the body assembly 210 may be respectively electrically connected to two conductive members 170 in the vaporizer 10, so as to form a power supply circuit to supply power to a linear vaporization core 130.

A mounting groove 211 is provided on the body assembly 210. A side of the vaporizer 10 close to the bottom wall of the base 140 may be inserted into the mounting groove 211 to be fixedly connected to the body assembly 210.

In summary, a person skilled in the art may easily understand that beneficial effects of this application are as follows: this application provides an electronic vaporization device and a vaporizer thereof. By providing a fine groove in an air inlet channel, condensed vaporized liquid in the air inlet channel may enter the fine groove for buffering, thereby preventing the condensed vaporized liquid from leaking out from an air inlet of the air inlet channel. Further, by providing the air inlet channel on either side of long sides of a cross section of a vaporization cavity, when a vaporization core is mounted in the vaporization cavity, a journey of passing a vaporization surface of the vaporization core performed by an airflow entering the vaporization cavity from the air inlet channel may be lengthened, thereby increasing a heating time of the airflow. In this way, condensate of vaporized liquid and splashed vaporized liquid generated in a vaporizer may be fully absorbed or heated and vaporized by the vaporization core, reducing occurrence of a phenomenon of inhalation leakage and condensate accumulation.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporizer, comprising:
a vaporization sleeve having a liquid storage cavity and a mounting cavity;
a mounting base arranged in the mounting cavity and forming a vaporization cavity, an air inlet channel and an air outlet channel being in communication with the vaporization cavity;
a vaporization core mounted in the vaporization cavity; and
a fine groove provided on an inner wall of the air inlet channel, the fine groove being configured to buffer liquid entering the air inlet channel.

2. The vaporizer of claim 1, wherein the mounting base comprises a base and a seal member, the seal member being arranged on a side of the base close to the liquid storage cavity, the seal member being engaged with the base and surrounding the base to form the vaporization cavity.

3. The vaporizer of claim 2, wherein an outer surface of a side wall of the base has an air guide groove, the air guide groove cooperating with a side wall of the mounting cavity to form the air inlet channel, an end of the air inlet channel close to the liquid storage cavity being in communication with the vaporization cavity, and an end away from the liquid storage cavity being in communication with external air, and
wherein the fine groove is provided in the air guide groove.

4. The vaporizer of claim 3, wherein the air guide groove extends along a side of the mounting base close to the liquid storage cavity to a side away from the liquid storage cavity, and the fine groove is provided on a bottom wall of the air guide groove and a width of the fine groove ranges from 0.2 mm to 1.2 mm.

5. The vaporizer of claim 3, wherein the air guide groove extends along a side of the mounting base close to the liquid storage cavity to a side away from the liquid storage cavity, and the fine groove is provided on a bottom wall of the air guide groove and a width of the fine groove ranges from 0.3 mm to 0.7 mm.

6. The vaporizer of claim 4, wherein a side of the air guide groove close to the liquid storage cavity has a first intercepting groove, and the fine groove is in communication with the vaporization cavity through the first intercepting groove.

7. The vaporizer of claim 6, wherein a side of the air guide groove away from the liquid storage cavity has a second intercepting groove, and the fine groove is in communication with the external air through the second intercepting groove.

8. The vaporizer of claim 4, wherein a middle portion of the air guide groove has at least one third intercepting groove, and the third intercepting groove separates the fine groove.

9. The vaporizer of claim 7, wherein the first intercepting groove comprises a first notch on an edge of the side wall of the base close to the liquid storage cavity, and the second intercepting groove comprises a second notch on an edge of a bottom wall of the mounting base.

10. The vaporizer of claim 2, wherein a baffle plate is arranged between the air inlet channel and the vaporization cavity,
wherein the baffle plate is configured to close the air inlet channel in a natural state so as to disconnect communication between the air inlet channel and the vaporization cavity, and
wherein the baffle plate is configured to open the air inlet channel when an inhaler inhales air from the vaporization cavity through the air outlet channel so as to communicate the air inlet channel with the vaporization cavity.

11. The vaporizer of claim 10, wherein a side wall of the seal member has an engagement groove sleeved on a side wall of the base, and
wherein a side wall on a side of the engagement groove is arranged between the air inlet channel and a side wall of the mounting cavity, and a side wall on an other side of the engagement groove is arranged between the vaporization core and the side wall of the base.

12. The vaporizer of claim 11, wherein the baffle plate is fixedly connected to a bottom wall of the engagement groove of the seal member, and the baffle plate is integrally formed with the seal member.

13. The vaporizer of claim 1, wherein a cross section of the vaporization cavity has two opposite long sides and two opposite short sides, and the air inlet channel is located on either side of the long sides.

14. An electronic vaporization device, comprising:
the vaporizer of claim 1, the vaporizer being configured to store to-be-vaporized liquid and vaporize the to-be-vaporized liquid to form an aerosol for inhalation by an inhaler; and
a body assembly configured to supply power to the vaporizer.
